(19) United States
(12) Patent Application Publication
(10) Pub. No.: US 2012/0172765 A1
(43) Pub. Date: Jul. 5, 2012

(54) Device and use of a pressure-sound-source for the treatment of fungal diseases

(76) Inventor: Cornelia Esenwein, Flensburg (DE)

(21) Appl. No.: 13/137,942

(22) Filed: Sep. 22, 2011

(30) Foreign Application Priority Data
Oct. 17, 2009 (DE) DE 10 2009 049 71

(51) Int. Cl. A61N 7/00

(52) U.S. Cl. 601/2

(57) ABSTRACT

The invention deals with a device and the use of an impulse-sound-source for the treatment of fungal diseases. The treatment of fungal diseases was until recently only possible through fungicides that cause patients strong side effects. The application of acoustic pressure waves in the diseased tissues heals mycoses without causing the mentioned side effects. The sound impulses have a fungicidal effect and ensure the dying off of the fungal colonies.

The following invention pertains to the use of a known device for shock wave therapy; its therapeutic head generates acoustic impulses that are induced in the human body.

Shock waves have been successfully applied since a long time for the treatment of kidney stones, gall stones and salivary calculus. The application of such shock wave sources are also well known in the orthopedic field, e.g., in the cases of delayed fracture-healing, shoulder calcifications, calcaneal spurs and tendon-based-inflammations. Shock waves are also applied in the wound healing field for the regeneration of tissues and enhancement of blood circulation of the ischemic tissue conditions, in addition to the formation of new blood vessels (WO 2005/075020), the treatment of cellulite (DE 19721218B4), treatment of heart muscles (EP 0929347), supporting stem cell differentiation and the stimulation of growth factors (VEGF, TGF-β, BMF). In addition, shock waves are shown to have an anti-bacterial effect (Gerdesmayer L., Ultrasound in Med. & Bio., Vol. 31, No. 1, pp. 115-119, 2005).

Different shock wave sources employ different generation principles to emit acoustic impulses. In this manner, electro hydraulic, electromagnetic, piezoelectric and ballistic systems can be used. A common characteristic is that these shock waves generate an acoustic pulse which is discharged either as a focused, unfocussed, planar or radial pressure wave or its pressure wave gets focused into a shock wave through an acoustic lens or a reflector or which possesses the characteristics of a shock wave already at the point of its generation.

The aim of the invention at hand is to set a new scope of utilization for acoustic pressure-impulse-source devices (pressure sound waves and shock waves) with or without focusing.

Based on this invention, the aim is achieved through the use of an established and known pressure-impulse-source with a therapy head which generates acoustic pulses that in turn are induced in the human body for the treatment of fungal diseases.

### TASKS/PROBLEM DEFINITION

Different skin and adnexa (nails, hair) diseases through fungus, fungoid and related pathogens (dermatomycosis) are very wide spread. A distinction is drawn between the dermatophytes (filamentous fungus), pathogenic saccharomycetes (yeast like fungi otherwise named candida) and mildew. Dermatophytes need organically bound carbon and grow with yarn-like vegetation called hyphae that form the myzel netting. The dermatophytes, like the mushrooms, tend to disperse in circular formations (fairy rings) on grass or in forests. The structural spread of mushrooms is also known as the biofilm problem; thus, it is a cross-linked organization of microorganisms that exhibit an increased resistance against medication due to their film formation. For example: the increased antibiotic resistance of the bacteria in the biofilm (amongst other things, through increased horizontal gene transfer, formation of persisters). The spores are difficult to combat and can survive for several years.

The dermatophytes are divided into trichophyton, epidermophyton and mikrosporum. The tricophyton have the highest adaptability and cause mycoses in haired heads, bearded area, between toes, foot soles and finger nails. In contrast, the epidermophyton arises almost solely on feet, groins, armpits and nails; it rarely afflicts the hair. The Microsporum types afflict the scalp hair, haired heads and body skin; they seldom afflict nails and skin folds.

Skin diseases caused by mushrooms, dermatomycoses are differentiated on the basis of the clinical changes they cause as follows:
1. Superficial forms with minor inflammatory reaction
   a. Tinea (Filamentous fungus diseases; pityriasis versicolor)
   b. Microspores (partly forms with substantial inflammatory reaction)
2. Forms with stronger inflammatory reaction
   a. Tinea corporis
   b. Tinea pedis
   c. Tinea inguinalis and axillaris (transitional forms may be of deep inflammatory reaction)
3. Forms that are also of deep inflammatory reaction
   a. Trichophytia Barbae
   b. Trichophytia Capillitii
   c. Tirchophytia of the lanugo-haired skin (superficial and deep forms)
4. Nail mycosis
5. Candida infections
6. Aphanomyces
7. Systematic mycoses

The currently known therapy is based on the localization of the disease either locally or systemically. The inventive treatment may be administered for all the above mentioned mycoses that manifest themselves locally on the body surface.

Consequently, aphanomyces and systemic mycoses are excluded. The invention presents an alternative and/or a supplement to the pharmacological treatment in the cases where the fungus is accessible for shock wave treatment on the body surface.

### APPLICATION EXAMPLE

The inventive application of pressure sound waves can be explained exemplarily through the example of the treatment of nail mycoses. The nail mycoses therapy is conducted according to the current medical professional standards, e.g., through the extraction or filing off of nails and/or the local or systemic administration of a fungicide over a long period of time (6-9/12 months). If the local application of a fungicide over a long period of time proves ineffective, then the systemic administration of a fungicide over a period of 6-9/12 months is the only last resort. In this case, a regular check-up must be conducted by a doctor due to the fact that the liver and kidney functions may be affected by the intake of medication over a longer period of time. Similarly, side effects like reactions of the muscle and skeleton system (myalgia, arthalgies) and allergic skin reactions (also to the mucous membrane) are common. All systemic medications are either unexplored or unsuitable for children and pregnant women. Similarly, this medication may not be prescribed for patients with signs of a ventricular dysfunction like a congestive heart failure or a known anamnestic heart insufficiency (other than life threatening infections).

The side effects are such that the patients would suffer of the disease for years with no alternative therapy.

The inventive application of pulse sources that emit acoustic pressure waves in the diseased tissues results in healing the nail mycoses without causing the side effects mentioned. The sound pulses possess a fungicidal effect and kill the fungus colonies. The fungi themselves, as well as their cross linked structure are reduced effectively through the shock waves such that the body is able to heal the infection through its own immune system. It was found that the linkage of the fungi that have built a biofilm and that were difficult to reach medically was reduced through the sound waves. The pathogenic, toxic effect that developed as a biofilm mycosis is sufficiently reduced through the application of shock waves such that it no longer surmounts the immune response of the body which in turn leads to healing. Through the additional administration of a local fungicide the efficacy of a drug is locally enhanced through the weakening of the biofilm such that the combination of both therapies may allow for a reduced dosage or duration of medication intake. The administration of the local fungicide can also take place through a bimixture of the fungicide in the coupling medium.

The treatment of the Trichophytia barbae or the Tinea inguinalis is administered according to the affected area. The shock waves are administered in measure to the affected area, such that the number of the required impulses increases in proportion to the surface area in question.

Additional advantages are that the enhanced metabolism and blood circulation that are caused by the pressure waves provide support to the body's immune defense mechanism against the fungi. In addition, the known stimulation of growth factors has a beneficial effect on the regeneration of the diseased tissues.

Sufficient and beneficial for the inventive, proposed application is the use of a focused electro hydraulic, electromagnetic or piezoelectric impulse-sound-source that releases shock waves in an energy flux density of 0.02 to 0.15 mJ/mm². A substantial number of the impulses, depending on the size of the area to be treated, lies between 200 and 400 impulses per cm² surface area.

In another design, unfocused impulse sound sources with acoustic pulses that do not necessarily dispose of the physical quality of shock waves yet are so high in energy, are applied such that the energy flux density reaches a range of 0.01 mJ/mm² to 0.08 mJ/mm². A substantial number of the impulses, depending on the size of the area to be treated, lies between 400 and 800 impulses per cm² surface area.

An advantageous design of the applicator for the treatment of fungal diseases is, in comparison to the conventionally known applicators of shock wave systems, substantially smaller and lighter with a rounded discharge opening of a maximum diameter of 20 mm. The application in podiatric therapy and outpatient care requires a small, compact and light device with a total weight of less than 3 Kg and a small, handy applicator. The advantages of the design are the functions for the setting of the energy parameter, frequency and the impulse trigger that are placed directly on the applicator such that only an extended high voltage generating transformer with a circuit without further control elements is needed together with a supply unit. The shock wave generation is carried out through the well-known generation system called: the piezoelectric, electromagnetic or electro-hydraulic generation technology.

### Case Study:

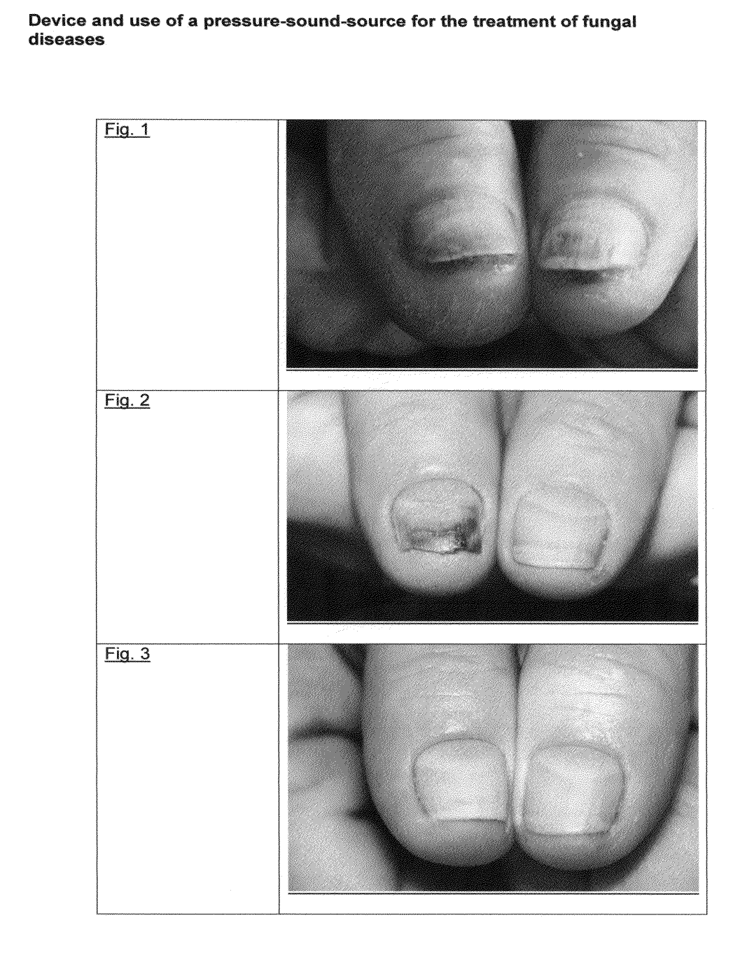

FIG. 1 shows the thumb of a 52 year old patient with nail mycosis on both thumbs. The mycosis was over two years old and had been treated with fungicides throughout the time period without any success. Both thumbs were subjected to a treatment of 1000 impulses with an energy-flow density of 0.06 mJ/mm² and a positive total energy of 1.4 mJ at a frequency of 5 Hz. The left thumb already healed after 5 weeks of the treatment (FIG. 2). The treatment of the right thumb was repeated after 6 weeks of the initial therapy using the same dosage and healed completely after an additional 8 weeks (FIG. 3).

FIG. 1 Nail mycosis on both thumbs before treatment

FIG. 2 Finger nails 5 weeks after first treatment

FIG. 3 Nail mycosis completely healed after second treatment after 14 weeks

1. The use of a known impulse-acoustic-source which possesses a therapy head for the generation of acoustic pulses that are then induced in the human body for the treatment of fungal diseases.

2. The use as mentioned in claim 1, whereby a non-focused, acoustic pulse is emitted by an impulse-sound-source.

3. Use as mentioned in claim 2, characterized in that an acoustic pulse with energy is emitted such that the impulses essentially exhibit the character of a shock wave.

4. Use as mentioned in claim 1, whereby a focused, acoustic pulse is emitted by an impulse-sound-source that generates shock waves when focussed.

5. Use as mentioned in claims 1 to 5, whereby the acoustic pulses possess energy of a minimum of 0.01 mJ and maximum of 0.25 mJ/mm², preferably 0.25 to 0.16 mJ/mm².

6. Use as mentioned in claims 1 to 5, whereby the number of acoustic pulses per treatment is between 200 and 800 impulses per cm².

7. Proceedings according to at least one of the previously mentioned claims that are characterized in that the coupling medium is a medical preparation with fungicides.

8. Applicator for carrying out the method according to at least one of the claims 1 to 4 mentioned that are characterized in that the applicator has a round outlet opening of less than 20 mm in diameter.

9. Applicator according to claim 7 that is characterized in that the regulation of the energy levels, frequency and impulse trigger is follows directly through a switch on the applicator.

10. Applicator according to claim 7 that is characterized in that the applicator is supplied directly by a small power supply unit with a transformer for high voltage generation without control elements.
